(19) United States
(12) Patent Application Publication
(10) Pub. No.: US 2026/0355525 A1
(43) Pub. Date: Nov. 12, 2026

(54) A 3D PRINTABLE PIEZOELECTRIC PLATONIC SOLIDS FOR OMNIDIRECTIONAL ULTRASONIC POWERING OF MINIATURE IMPLANTS

(71) Applicant: University of South Florida, Tampa, FL (US)

(72) Inventor: Albert KIM, Tampa, FL (US)

(21) Appl. No.: 19/129,222

(22) PCT Filed: Nov. 10, 2023
(86) PCT No.: PCT/US2023/079360

§ 371 Date: May 12, 2025

Related U.S. Application Data
(60) Provisional application No. 63/383,207, filed on Nov. 10, 2022.

(51) Int. Cl. A61N 1/372; B06B 1/06; B33Y 10/00; B33Y 70/10; B33Y 80/00; H10N 30/045; H10N 30/077; H10N 30/092; H10N 30/85

(52) U.S. Cl. CPC A61N 1/37217; B06B 1/0644; B33Y 70/10; B33Y 80/00; H10N 30/045; H10N 30/077; H10N 30/092; H10N 30/852; B06B 2201/76; B33Y 10/00

(57) ABSTRACT

A device comprising a piezoelectric solid and a miniaturized electronic device; wherein the piezoelectric solid comprises a polymer and metal oxide nanoparticles and has a platonic solid shape; wherein a face of the piezoelectric solid comprises a concentric void; and wherein the miniaturized electronic device is placed inside the concentric void.

### CROSS REFERENCE TO RELATED APPLICATIONS

This application claims the benefit of priority to U.S. Provisional Application 63/383,207, filed Nov. 10, 2022, which is incorporated by reference herein in its entirety.

### ACKNOWLEDGEMENT OF GOVERNMENT FUNDING

This invention was made with government support under Grant No. 2143723 awarded by the National Science Foundation. The government has certain rights in the invention.

### BACKGROUND

Since the successful clinical implementation of the pacemaker in the 1960s (Greatbatch, W., 1979. Cardiac pacemaker. U.S. Pat. No. 4,157,720 A) (Greatbatch, W., 1962. Medical cardiac pacemaker. U.S. Pat. No. 3,057,356 A), subsequent decades have witnessed abundant implantable medical devices (IMDs). Leveraging advances in nano and microelectromechanical systems (MEMS), integrated circuits, wireless communication, and battery technologies (House, W. F., 1976. Cochlear implants. Annals of Otology, Rhinology & Laryngology 85, 3-3) (Mackay, R. S., et al., 1957. Endoradiosonde. Nature 179, 1239-1240) (Rogers, J. A., et al., 2010. Materials and mechanics for stretchable electronics. science 327, 1603-1607) (Seo, D., et al., 2016. Wireless recording in the peripheral nervous system with ultrasonic neural dust. Neuron 91, 529-539), the IMDs indeed opened the new era of modern healthcare. One recent example is the brain-machine interface via a neural implant (equip with thousands of microelectrodes, a processor, and often with wireless communication), which is soon expected to assist, augment, or repair human cognitive or sensory-motor functions in precise control (Normann, R. A., et al., 2016. Clinical applications of penetrating neural interfaces and Utah Electrode Array technologies. Journal of Neural Engineering 13).

A fundamental requirement for the successful operation of these IMDs is an adequate and reliable power supply. Two significant advances in the energy source area in this period were the appearance of lithium batteries and inductive powering (Scrosati, B., et al., Hassoun, J., 2013. Lithium batteries: advanced technologies and applications. John Wiley & Sons) (van Schuylenbergh, K., et al., 2009. Inductive Powering: Basic Theory and Application to Biomedical Systems. Springer). Wilson Greatbatch, the pioneer entrepreneur and inventor of the cardiac pacemaker, was the first to develop and commercialize lithium batteries for implantable medical devices (Greatbatch, W., et al., 1971. The solid-state lithium battery: a new improved chemical power source for implantable cardiac pacemakers. Biomedical Engineering, IEEE Transactions on 317-324) (Holmes, C., 1997. The Bourner Lecture: electrochemical power sources—an important contributor to modern health care. Journal of power sources 5). This resulted in a significant increase in the device lifetime and thus minimize the number of surgeries for battery replacement. Inductive powering offers an alternative approach for devices that cannot utilize primary batteries, mainly due to large power consumption (cochlear implants) or limited anatomical space (intra-ocular implants). Inductive powering has reached a mature status and has dominated wireless IMDs for the past five decades. However, there are some practical issues, including short working distance, high misalignment sensitivity, tissue heating, and difficulties in fabricating sub-mm coils with adequate inductance and quality factors.

Acoustic (or ultrasonic) powering has been explored as an effective solution to address the challenges as mentioned earlier, especially for millimeter-scale implantable devices (Campbell, R., et al., 2021. Implantable Cisplatin Synthesis Microdevice for Regional Chemotherapy. Adv. Healthcare Mater. 10, 2001582) (Kim, A., et al., 2019. An Implantable Ultrasonically-Powered Micro-Light-Source (μLight) for Photodynamic Therapy. Sci Rep 9, 1395) (Maleki, T., et al., 2011. An Ultrasonically Powered Implantable Micro-Oxygen Generator (IMOG). IEEE Trans. Biomed. Eng. 58, 3104-3111) (Shi, C., et al., 2021. Application of a sub-0.1-mm 3 implantable mote for in vivo real-time wireless temperature sensing. Sci. Adv. 7, eabf6312) (Song, S. H., et al., 2015. Omnidirectional Ultrasonic Powering for Millimeter-Scale Implantable Devices. IEEE Trans. Biomed. Eng. 62, 2717-2723) (Sonmezoglu, S., et al., 2021. Monitoring deep-tissue oxygenation with a millimeter-scale ultrasonic implant. Nat Biotechnol). It is achieved through the conversion of the mechanical energy propagating through the body in the form of ultrasound into electrical energy via an ultrasonic transducer. When ultrasonic waves propagate through the body, a deeply seated microdevice can pick up a portion of them, converting them into electrical power via a piezoelectric receiver. This electrical power can operate various life-sustaining functions such as sensing, actuation, or chemical delivery. Ultrasonic powering offers overarching advantages of long-distance operation and high efficiency with an mm-scale receiver. For instance, with a 2-mm receiver placed 10 cm away from the transmitter, the efficiency of ultrasonic powering was the orders of magnitude higher than inductive powering (0.02% vs. 0.00002%, respectively) (Denisov, A., et al., 2010. Ultrasonic vs. Inductive Power Delivery for Miniature Biomedical Implants. 2010 International Conference on Body Sensor Networks 84-89).

Even with these advantages, a misalignment challenge remains in ultrasonic powering; the powering efficiency can be significantly reduced if the incoming ultrasound direction is not orthogonal to the receiving surface, which may abruptly disturb the continuous operation. This limitation originates from the geometric constrains; ultrasonic receivers often take a form factor of cuboids or discs with the poling vector in one direction between two parallel planes of electrodes. For the highest energy conversion efficiency, the incident ultrasonic waves must interface the receiver surface parallelly (i.e., orthogonal to the poling vector). However, when powering miniature implants, these typical form factors can be less than ideal; the propagating waves are often deflected within the path (due to inhomogeneous acoustic media) and the miniature implants can shift and rotate, resulting in a significant misalignment. A millimeter-sized symmetric cuboidal form factor receiver could improve the orientation misalignment sensitivity due to Poisson's ratio; when ultrasonic waves hit the lateral sides of the receiver, the longitudinal deformation also contributes to the power generation (Song, S. H., et al., 2015. Omnidirectional Ultrasonic Powering for Millimeter-Scale Implantable Devices. IEEE Trans. Biomed. Eng. 62, 2717-2723). However, the misalignment effects were still pronounced, especially when the incoming waves were being scattered off the vertices and edges. This coupled with the fact that a millimeter-scale receiver is often designed for deep body implantation where the alignment between the transmitter and receiver is exceptionally challenging, the overall electrical power outputs were often greatly reduced. Additionally, many of reported ultrasonic powering use lead zirconate titanate (PZT) (Shi, C., et al., 2021. Application of a sub-0.1-mm 3 implantable mote for in vivo real-time wireless temperature sensing. Sci. Adv. 7, eabf6312) (Sonmezoglu, S., et al., 2021. Monitoring deep-tissue oxygenation with a millimeter-scale ultrasonic implant. Nat Biotechnol), which raises significant safety concerns due to the lead poisoning. What are thus needed are new omnidirectional ultrasonic receivers that can address the challenges of current receivers. The disclosed compositions, methods, and devices address these and other needs.

### SUMMARY

Ultrasound has recently been explored as an attractive source of power, especially for deeply seated implantable microdevices. However, the receiver has remained as a slab of bulk piezoelectric materials, which is not an ideal form factor for wireless power transfer. Disclosed herein is an omnidirectional ultrasonic powering featuring three-dimensional polyhedral receivers. Based on a 3D printing technique of lead-free barium titanate ceramic, four highly symmetric miniaturized regular polyhedral, known as Platonic solids (i.e., cube, octahedron, dodecahedron) and a sphere are disclosed. By modifying each geometry, axial and radial poling, output power levels, efficiency, and angular sensitivity, the Platonic solids can exhibit superior power transfer efficiency with excellent omnidirectionality. The 3D printability of the Platonic solid also allows customizable packaging, which can be implemented as an implantable micro light source. Overall, the proposed ultrasonic powering scheme can be a translational solution for many implantable devices.

In an aspect, provided is a piezoelectric solid comprising a polymer and metal oxide nanoparticles, wherein the piezoelectric solid is has a platonic solid shape.

In another aspect, provided is a device comprising a piezoelectric solid and a miniaturized electronic device; wherein the piezoelectric solid comprises a polymer and metal oxide nanoparticles and has a platonic solid shape; wherein a face of the piezoelectric solid comprises a concentric void; and wherein the miniaturized electronic device is placed inside the concentric void.

In another aspect, provided is an ultrasonic receiver comprising any of the disclosed devices. In another aspect, provided is an implantable microdevice comprising any of the disclosed ultrasonic receivers, wherein the ultrasonic receiver transfers power to the implantable microdevice via the miniaturized electronic device.

In another aspect, provided is a method of making a piezoelectric solid, comprising: a) preparing a binder solution comprising a polymer and a binder; b) adding metal oxide nanoparticles to the binder solution; c) additively manufacturing the binder solution into a platonic solid shape; d) debinding and sintering the platonic solid to form a piezoelectric solid; and e) poling the piezoelectric solid.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRA WINGS

The accompanying figures, which are incorporated in and constitute a part of this specification, illustrate several aspects described below.

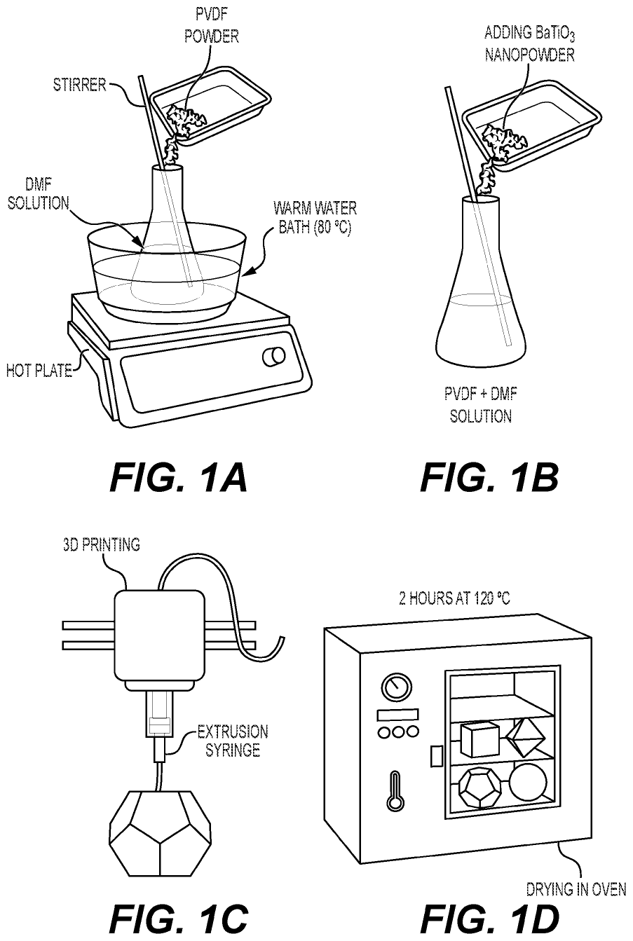

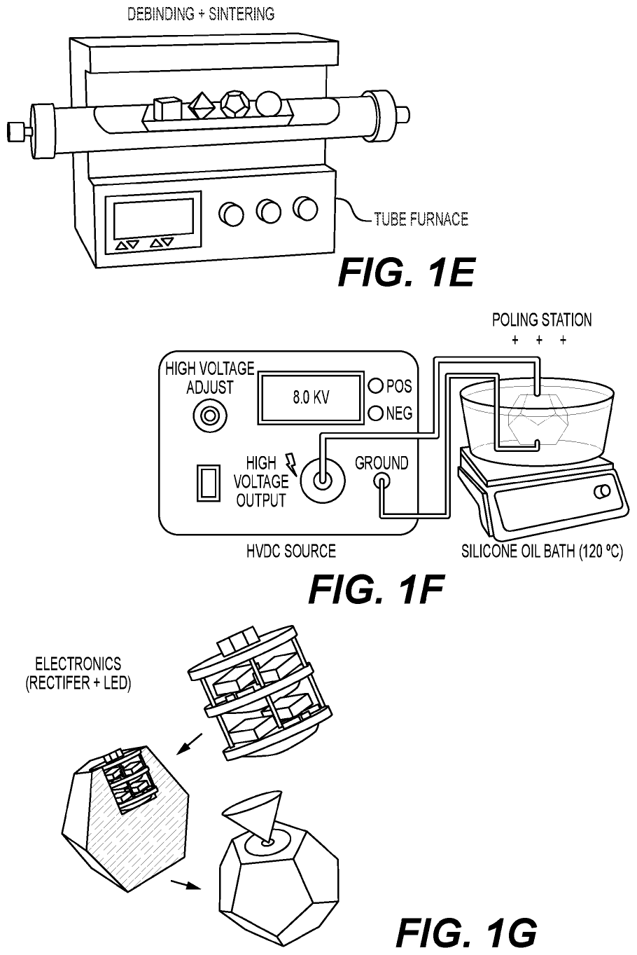

FIGS. 1A-1G depict a fabrication process. FIG. 1A shows PVDF binder added to DMF solution. FIG. 1B shows BaTiO₃ nanoparticles added. FIG. 1C shows 3D extrusion printing of Platonic solids. FIG. 1D shows evaporating DMF in an oven. FIG. 1E shows debinding and sintering in a tube furnace. FIG. 1F shows poling. FIG. 1G shows assembling electronics.

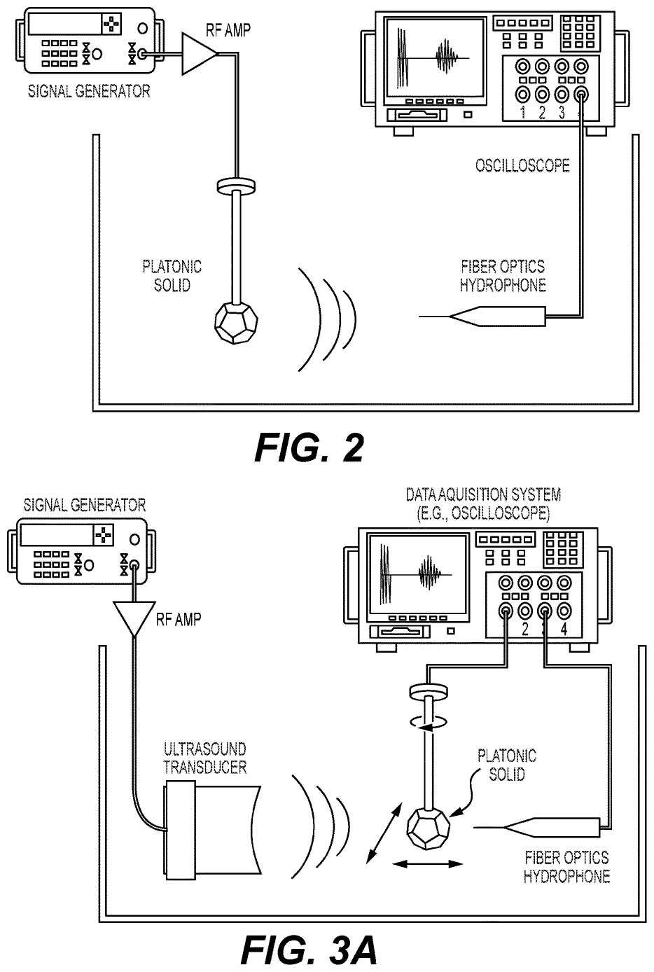

FIG. 2 depicts an experimental setup for a resonant frequency study.

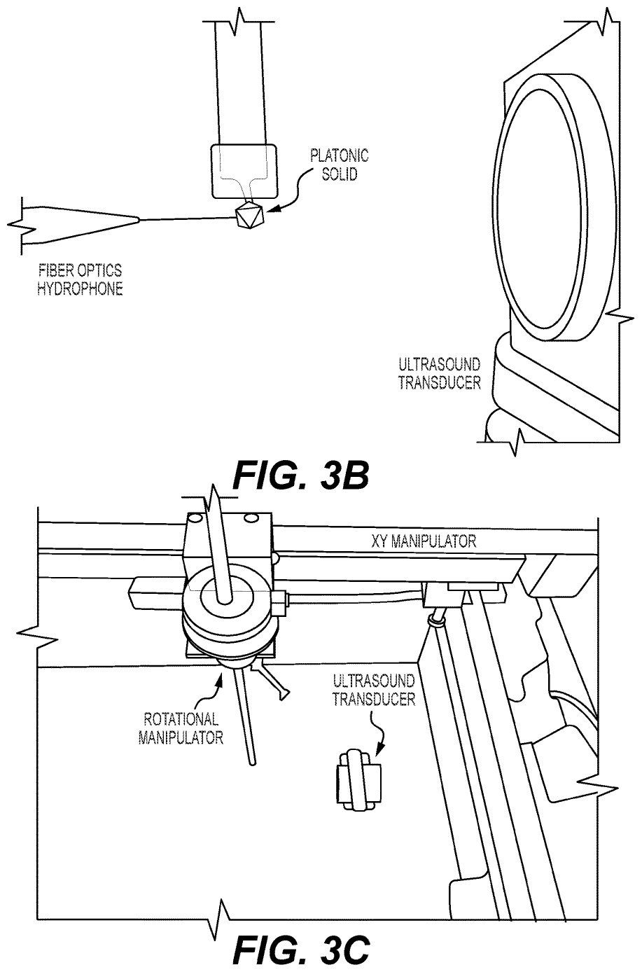

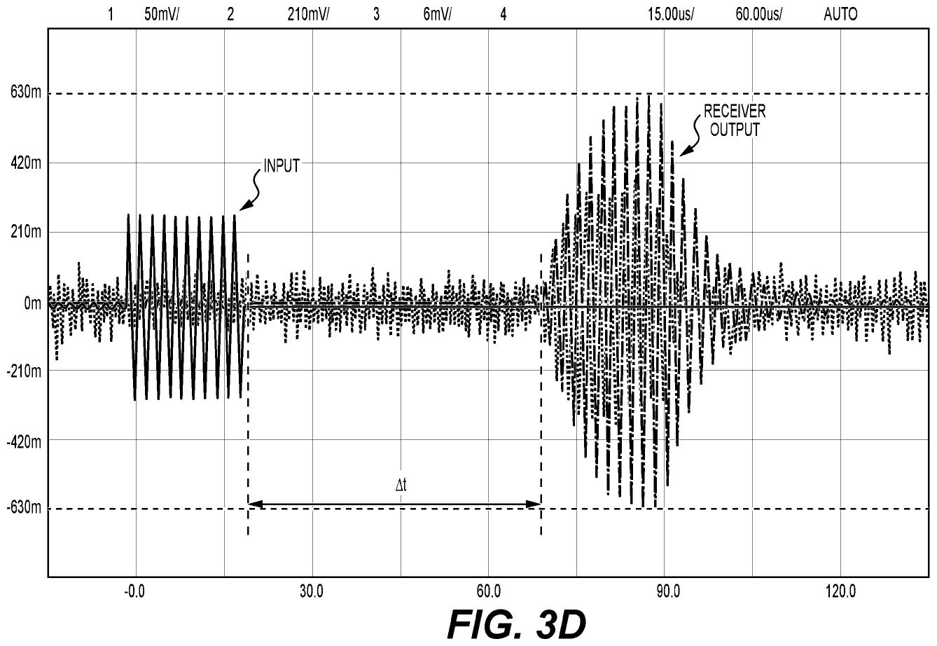

FIGS. 3A-3D depict ultrasonic powering characterization. FIGS. 3A-3C show an experimental setup and FIG. 3D shows an example of input and output waves.

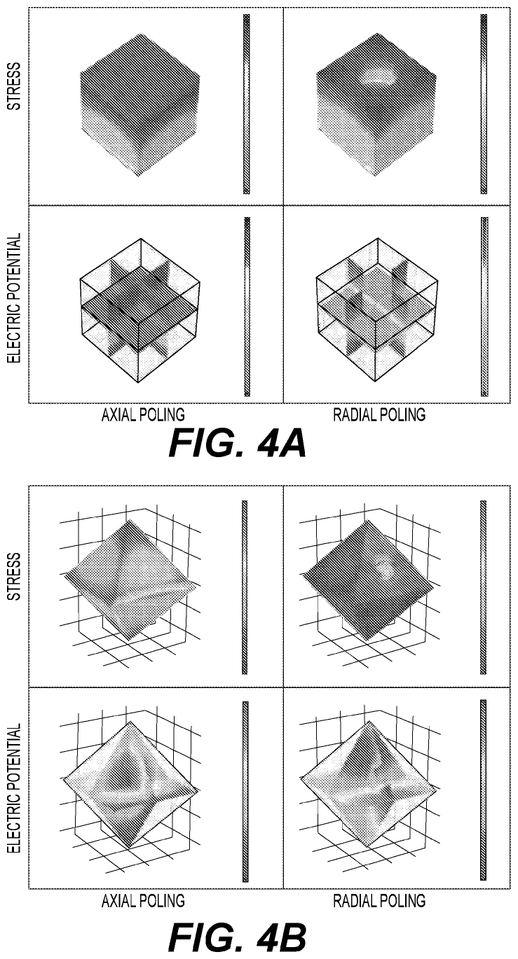

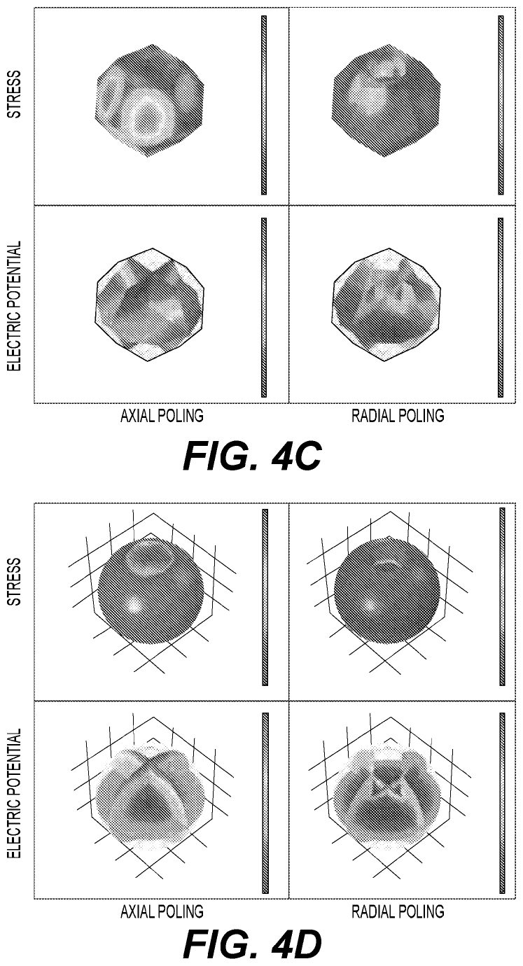

FIGS. 4A-4D depict a COMSOL simulation. Axial and radial poling clears redistribute the stress, thereby shifting the resonant frequency.

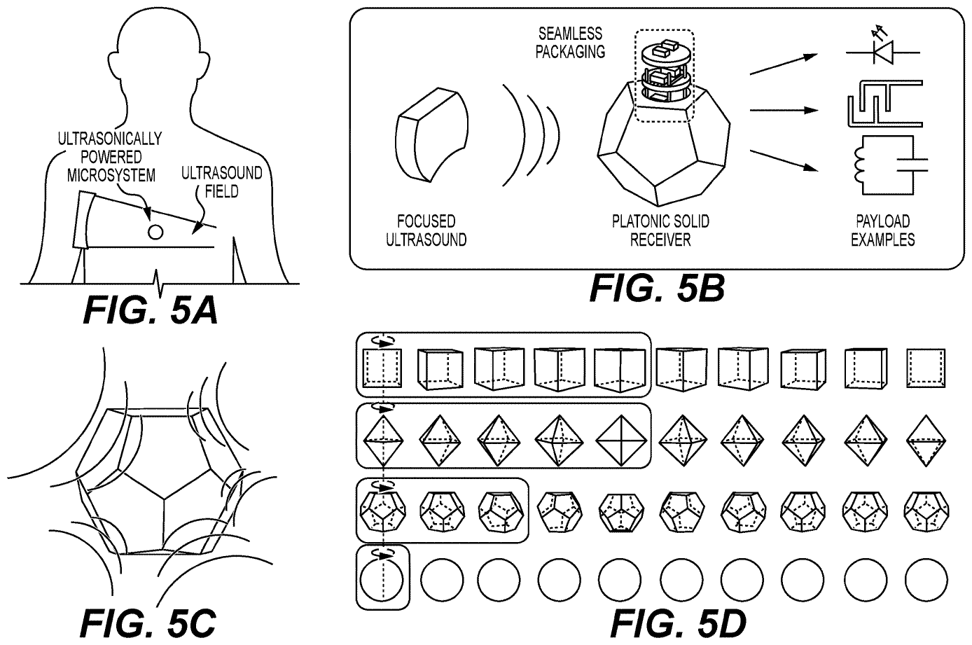

FIGS. 5A-5D depict ultrasonic powering for miniature implants. FIG. 5A shows how a deeply seated implant picks up a portion of ultrasound energy provided by an external transmitter. FIG. 5B shows how the converted electrical energy can be used to power implantable devices for applications such as sensing, actuation, and chemical delivery. Platonic solid can also be modified and provide an inner space for a seamless packaging of electronics such as a power recovery circuit (i.e., rectifier) and other loads. FIG. 5C shows how the ultrasonic powering can be omnidirectional when a Platonic solid design is implemented as a receiver (dodecahedron is shown as an example): Platonic solids have a greater number of faces orthogonal to the incident waves. FIG. 5D shows how the effective area is maintained even when it is rotated due to its high periodicity. For example, the effective areas are repeated every 45°, 30°, and 18° for cube, octahedron, and dodecahedron, respectively.

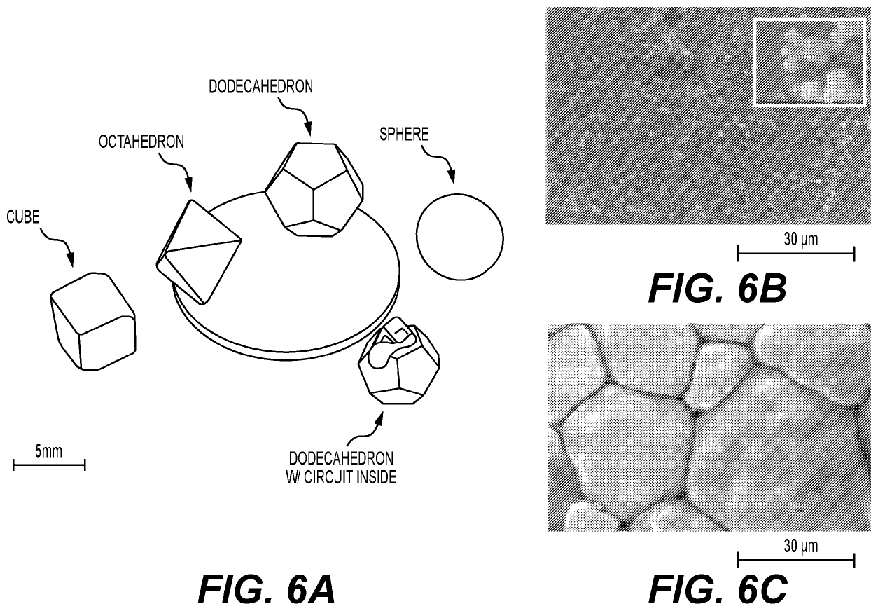

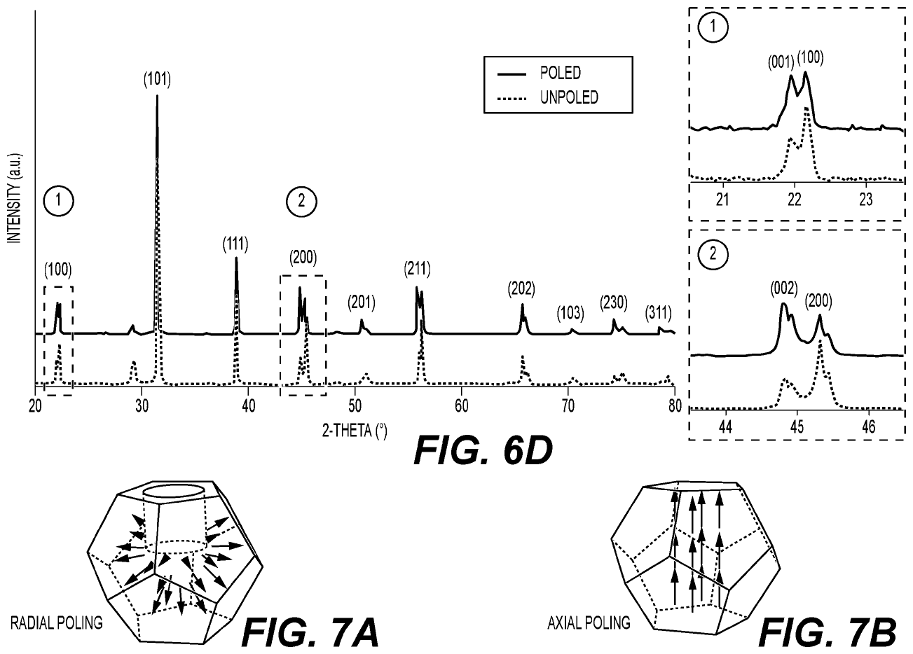

FIGS. 6A-6D depict a platonic solid receiver. FIG. 6A shows fabricated polyhedral ultrasound transducers: cube, octahedron, dodecahedron, and sphere. Dodecahedron with an embedded circuit is also shown in the right bottom corner. FIG. 6B shows SEM pictures after binding, and FIG. 6C shows SEM pictures after sintering. FIG. 6D shows an X-ray diffraction pattern: peaks splitting at 22° (100/001) and 45° (200/002) indicates the piezoelectricity due to poling.

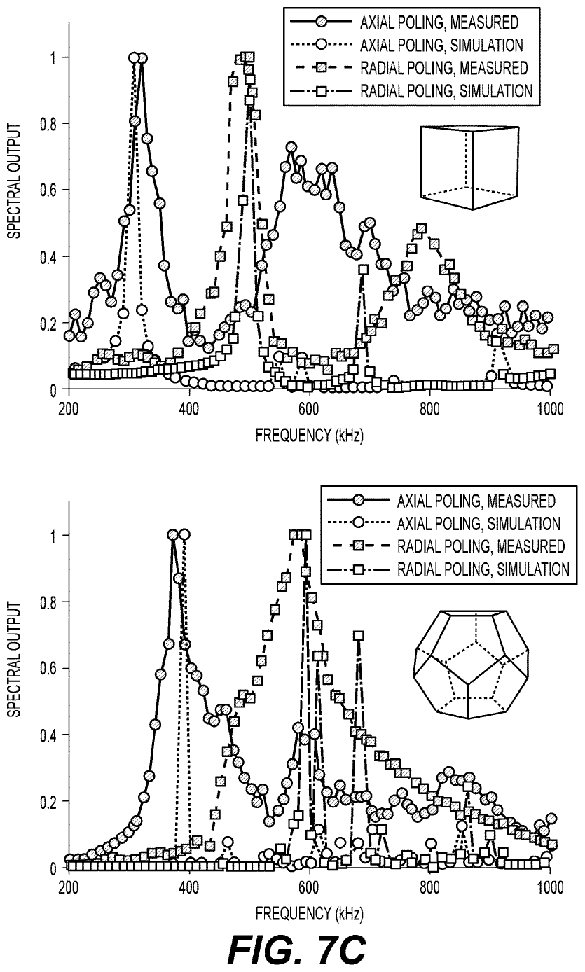

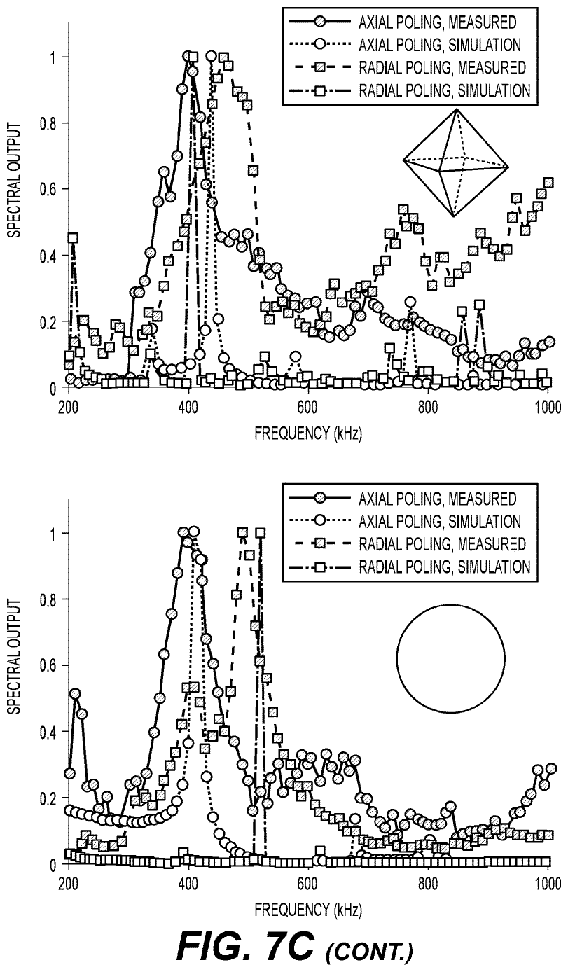

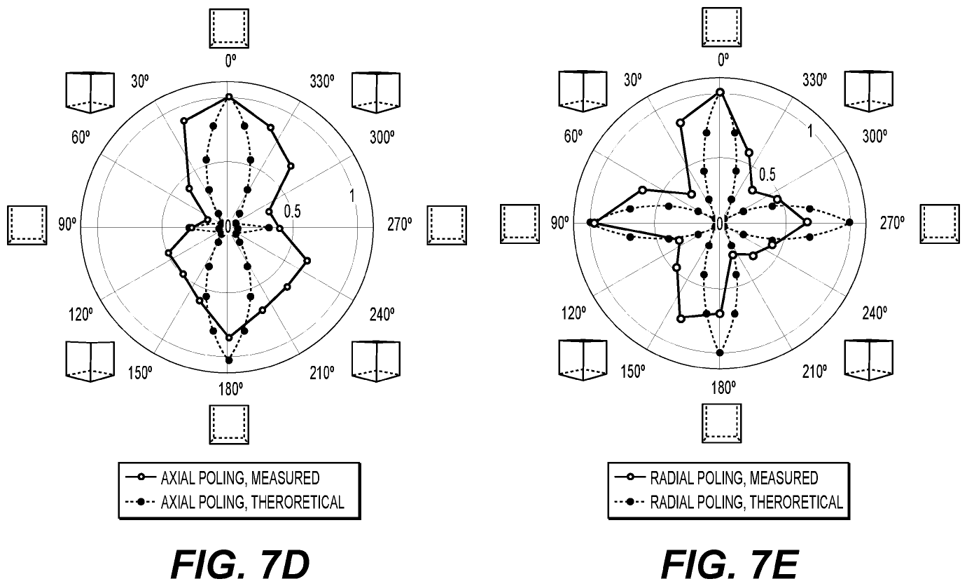

FIGS. 7A-7E depict the effect of poling direction. FIGS. 7A-7B are illustrations of radial (FIG. 7A) and axial (traditional) (FIG. 7B) poling, and FIG. 7C shows measured and simulated resonant frequencies of cube, octahedron, dodecahedron, and sphere. FIGS. 7D-7E show angular response of axially poled cube (FIG. 7D) and radially poled cube (FIG. 7E); the radial poling improves d₃₃ response.

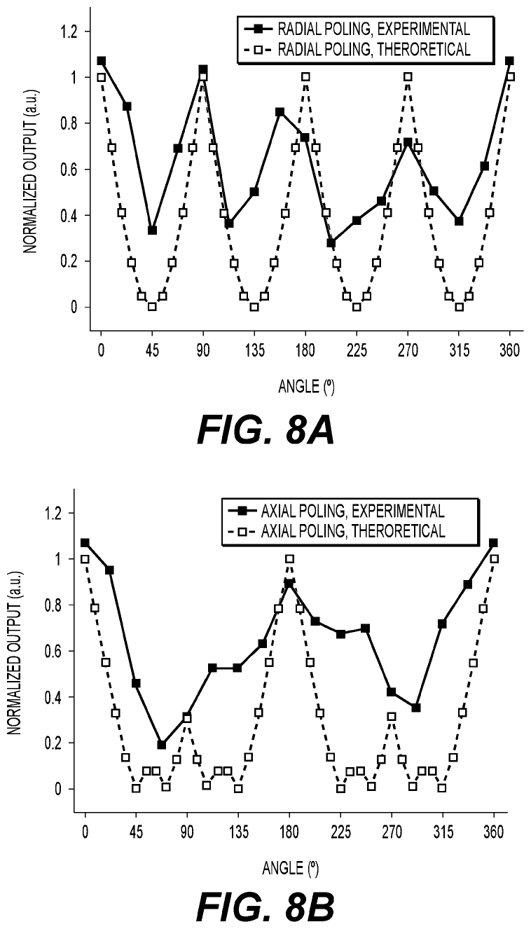

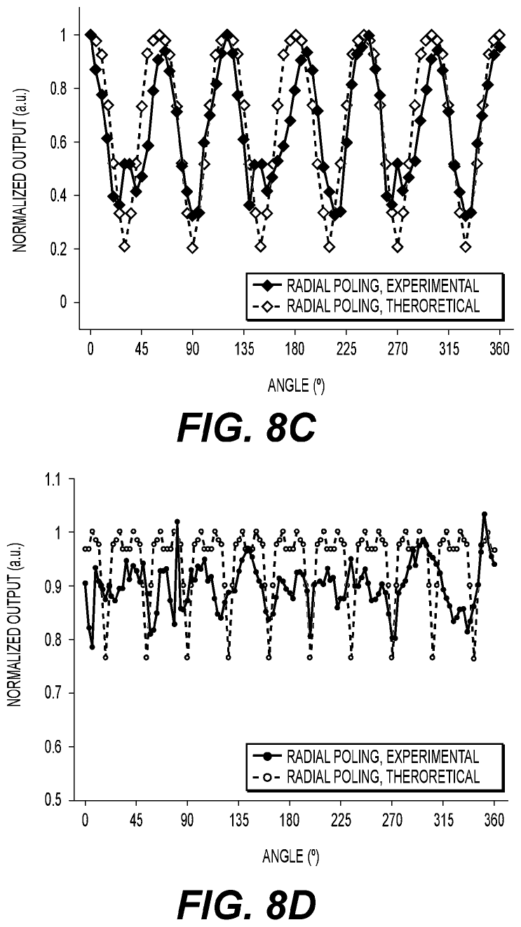

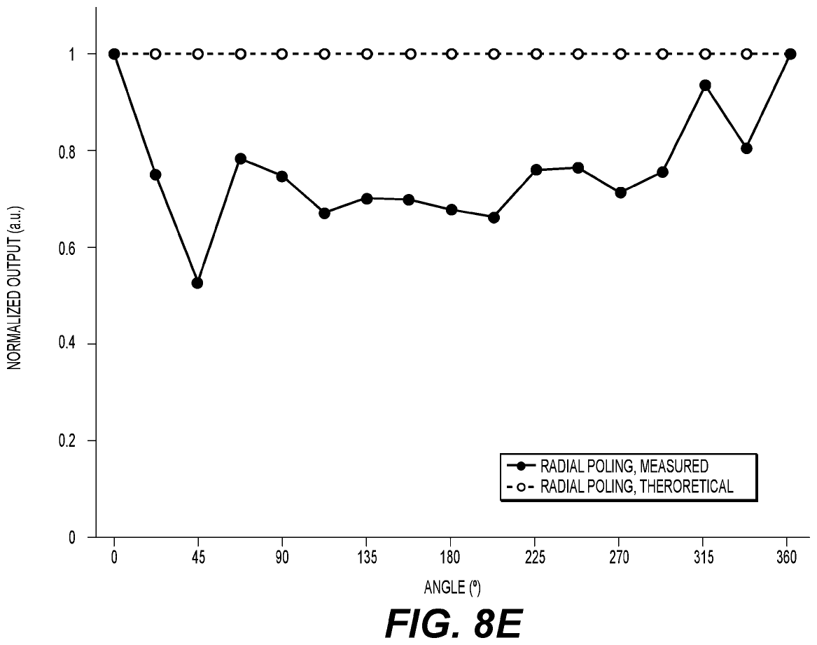

FIGS. 8A-8E depict orientation misalignment response of Platonic solid receiver for cube (radial poling) (FIG. 8A), cube (axial poling) (FIG. 8B), octahedron (FIG. 8C), dodecahedron (FIG. 8D), and sphere (FIG. 8E).

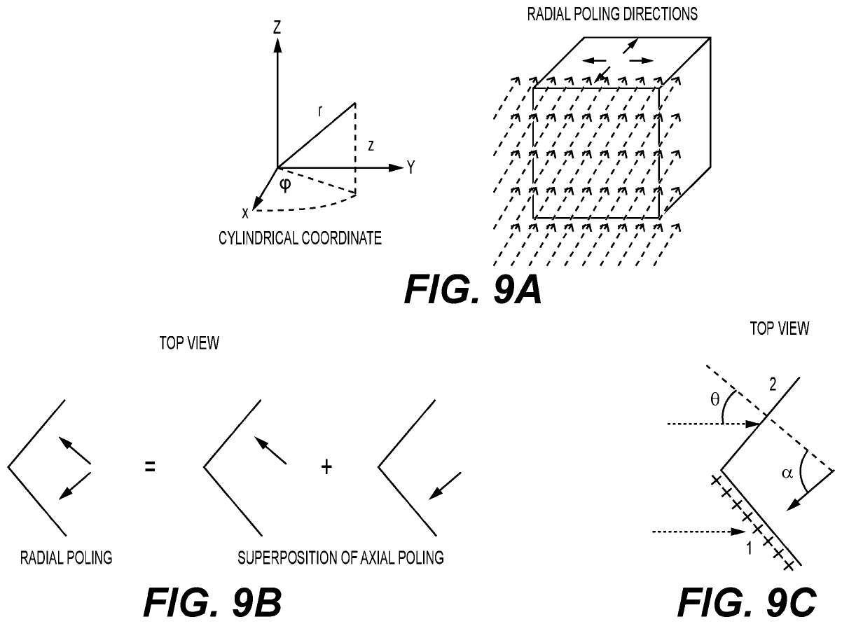

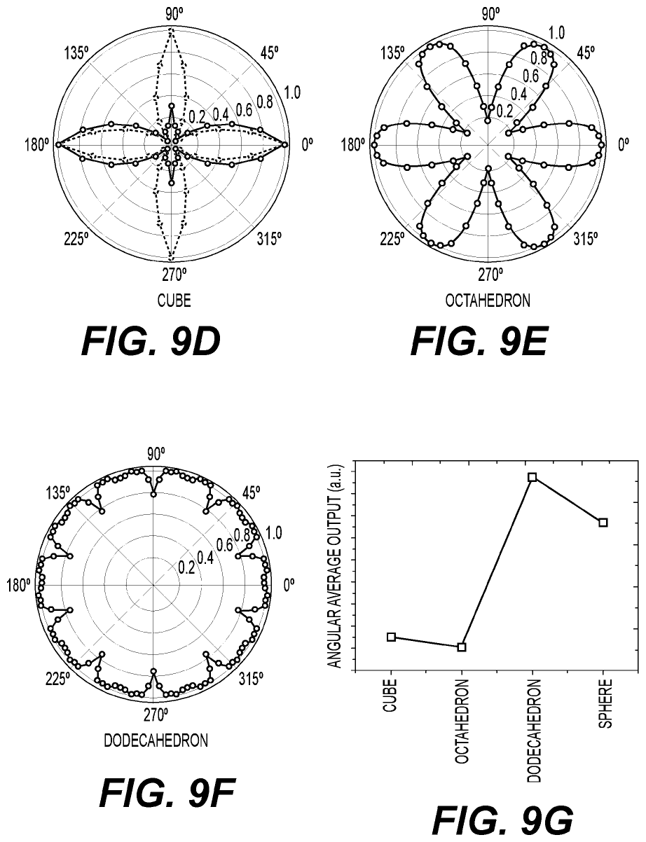

FIGS. 9A-9G depict theoretical calculations for angular responses of different radially poled Platonic solids. FIG. 9A shows the calculation setup. FIG. 9B shows the decomposition of radial poling into axial poling vectors. FIG. 9C shows the theoretical calculation rationale. FIGS. 9D-9F show polar plots for normalized angular response of cube, octahedron, and dodecahedron, respectively. FIG. 9G shows the theoretical angle average voltage response.

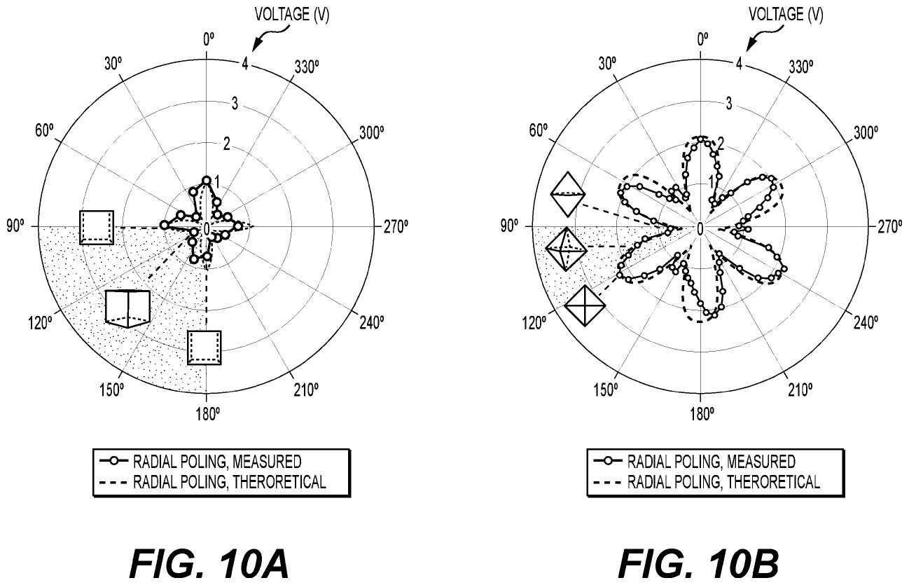

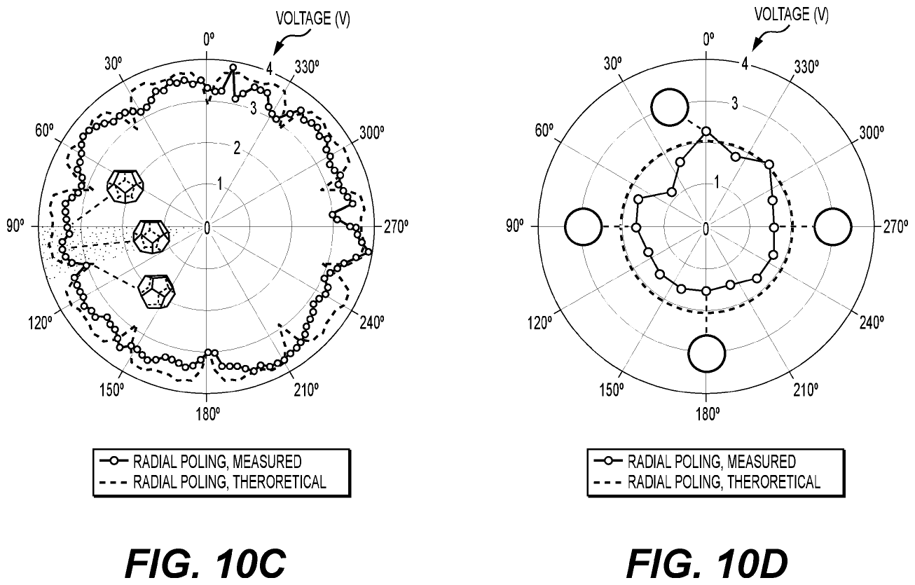

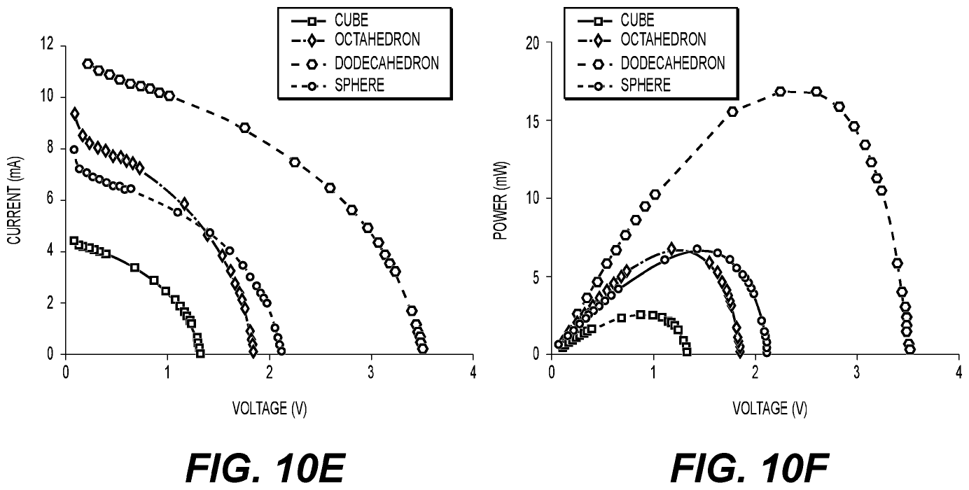

FIGS. 10A-10F depict orientation misalignment response of Platonic solid receivers: cube (capacitance=276.2 pF) (FIG. 10A), octahedron (capacitance=207.0 pF) (FIG. 10B), dodecahedron (capacitance=329.31 pF) (FIG. 10C), and sphere (capacitance=284.29 pF) (FIG. 10D). Overall a dodecahedron exhibited omnidirectional orientation with superior power transfer efficiency. FIG. 10E shows measured I-V 15 characteristics and FIG. 10F shows its associated power density.

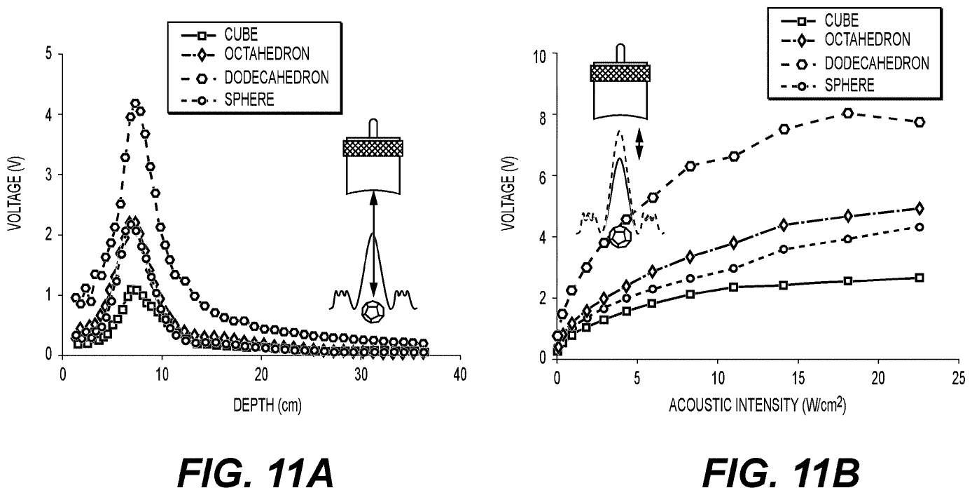

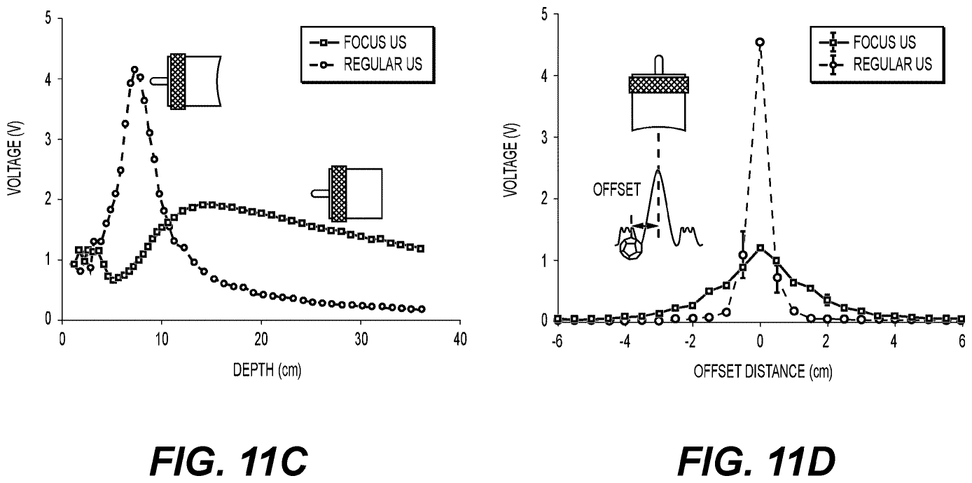

FIGS. 11A-11D depict ultrasonic powering performance of Platonic solids. FIG. 11A shows operation depth. FIG. 11B shows input intensity responses. FIGS. 11C-11D show operation depth and offset depending on the type of ultrasound: focused ultrasound and regular ultrasound.

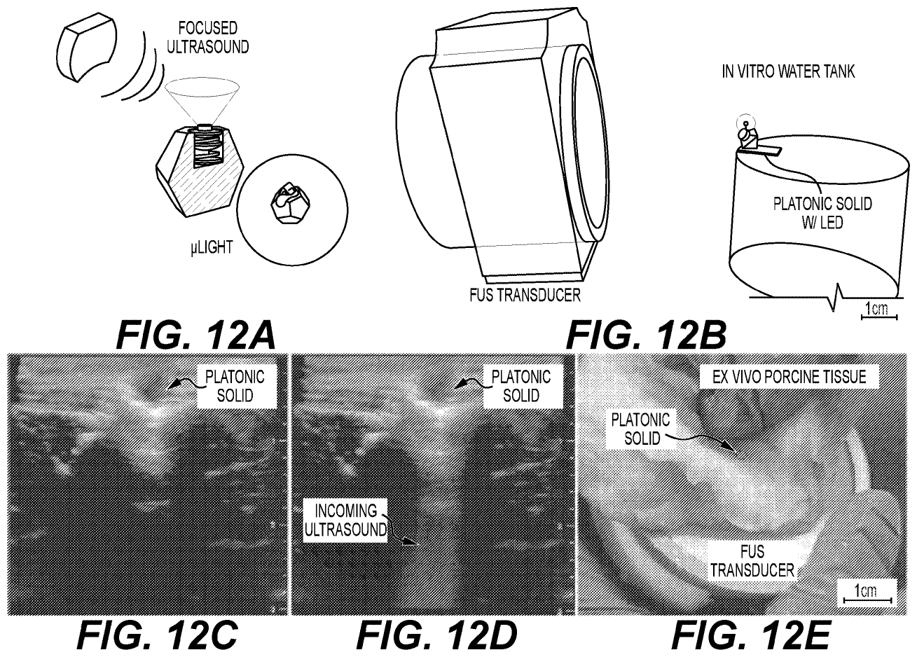

FIGS. 12A-12E depict a demonstration of Platonic solid as an implantable micro light source (μLight). FIG. 12A shows the design of μLight that contains electronics in the concentric void of the Platonic solid. FIG. 12B shows blue LED testing in a water bath in vitro. FIG. 12C shows ultrasonic imaging after the implantation and FIG. 12D shows ultrasonic imaging during operation (incoming ultrasonic waves from the FUS transducer can be seen under the imaging). FIG. 12E shows red LED light irradiance from the Platonic solid through the porcine tissue.

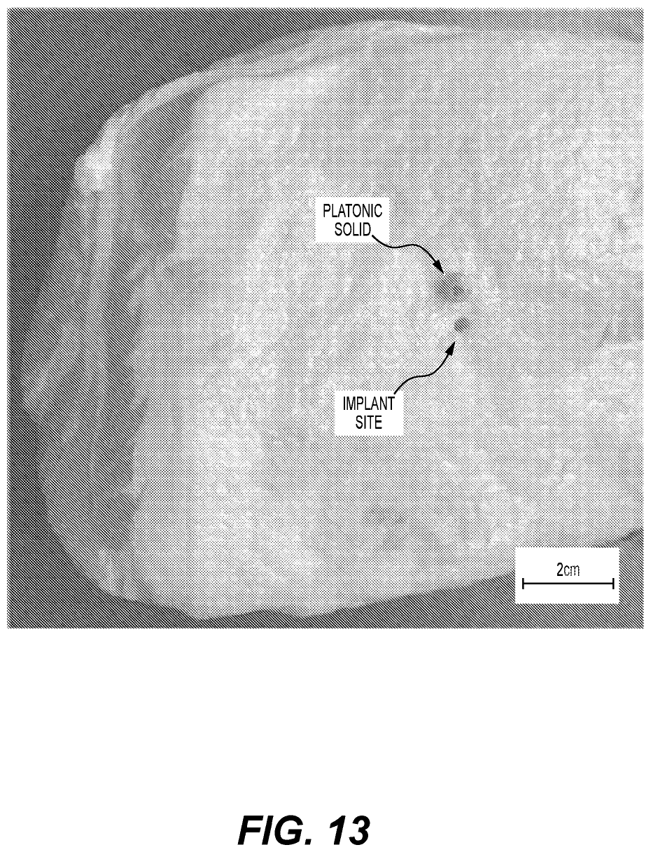

FIG. 13 depicts a picture of porcine tissue during implantation.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### DEFINITIONS

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to “a solid” includes aspects having two or more such solids unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise. DEVICE

In an aspect, provided is a piezoelectric solid comprising a polymer and metal oxide nanoparticles, wherein the piezoelectric solid is has a platonic solid shape. A “piezoelectric” solid is a solid that can generate electrical charge under mechanical stress.

As used herein, the term “platonic solid shape” refers to 3D shape having congruent, regular polygonal faces. In a platonic solid, the same number of faces meet at each vertex. There are five such solids: a tetrahedron, a cube, an octahedron, a dodecahedron, and an icosahedron.

In some aspects, the metal oxide is BaTiO₃, Pb(Zrₓ,Ti₁₋ₓ)O₃, PbTiO₃, ZnO, or combinations thereof. In some aspects, the metal oxide nanoparticles have a diameter from about 200 to about 800 nm, including exemplary ranges of from about 250 nm to about 750 nm, from about 300 nm to about 700 nm, from about 350 nm to about 650 nm, from about 400 nm to about 600 nm, from about 450 nm to about 550 nm, from about 200 nm to about 400 nm, from about 250 nm to about 350 nm, from about 600 nm to about 800 nm, or from about 650 nm to about 750 nm.

In some aspects, the polymer is a thermoplastic. As used herein, the term “thermoplastic” refers to a polymer that be molded when heated and cooled into a solid shape. In some aspects, the polymer is polyvinyl fluoride, acrylonitrile butadiene styrene, polycarbonate, polyethylene, polypropylene, polyvinyl chloride, or combinations thereof.

In some aspects, the piezoelectric solid has a tetrahedron shape, a cube shape, an octahedron shape, a dodecahedron shape, or an icosahedron shape.

In some aspects, the piezoelectric solid is axially poled. In some aspects, the piezoelectric solid is radially poled. A piezoelectric solid is made up of small crystalline or semicrystalline units, each of which have their own dipole moment (e.g., electrical charge gradient). “Poling” a piezoelectric solid refers to using an external electromagnetic field (e.g., from a magnet and/or electric current) to force all the dipole moments to align. Most conventionally, piezoelectric solids are “axially poled”—the dipole moments are aligned along an axis of the solid. However, the piezoelectric platonic solids described herein can be “radially poled”—the dipole moments are aligned radially from the center of the solid to the surface.

In some aspects, the piezoelectric solid further comprises a concentric void on a face of the piezoelectric solid. The concentric void can be etched or carved out from the solid to provide a divot that allows access to the interior of the piezoelectric solid. In some aspects, the concentric void can be cylindrical, rectangular, pyramidal, tapered, or another suitable shape.

In some aspects, the concentric void has a diameter of from about 1 mm to about 3 mm, or from about 1.25 mm to about 2.75 mm, or from about 1.5 mm to about 2.5 mm, or from about 1.75 mm to about 2.25 mm, or from about 1 mm to about 1.75 mm, or from about 1.25 mm to about 1.5 mm, or from about 2.25 mm to about 3 mm, or from about 2.5 mm to about 2.75 mm, or from about 1 mm to about 2 mm. In some aspects, the concentric void has a depth of from about 1 mm to about 3 mm, or from about 1.25 mm to about 2.75 mm, or from about 1.5 mm to about 2.5 mm, or from about 1.75 mm to about 2.25 mm, or from about 1 mm to about 1.75 mm, or from about 1.25 mm to about 1.5 mm, or from about 2.25 mm to about 3 mm, or from about 2.5 mm to about 2.75 mm, or from about 1 mm to about 2 mm.

In some aspects, the piezoelectric solid has an edge length of from about 2.5 mm to about 7 mm, or from about 2.75 mm to about 6.75 mm, or from about 3 mm to about 6.5 mm, or from about 3.25 mm to about 6.25 mm, or from about 3.5 mm to about 6 mm, or from about 3.75 mm to about 5.75 mm, or from about 4 mm to about 5.5 mm, or from about 4.25 mm to about 5.25 mm, or from about 4.5 mm to about 5 mm, or from about 2.5 mm to about 4.5 mm, or from about 2.75 mm to about 4.25 mm, or from about 3 mm to about 4 mm, or from about 3.25 mm to about 3.75 mm, or from about 5 mm to about 7 mm, or from about 5.25 mm to about 6.75 mm, or from about 5.5 mm to about 6.5 mm, or from about 5.75 mm to about 6.25 mm.

In some aspects, the piezoelectric solid has a surface area of from about 100 mm² to about 200 mm², or from about 110 mm² to about 190 mm², or from about 120 mm² to about 180 mm², or from about 130 mm² to about 170 mm², or from about 140 mm² to about 160 mm², or from about 100 mm² to about 150 mm², or from about 110 mm² to about 140 mm², or from about 120 mm² to about 130 mm², or from about 150 mm² to about 200 mm², or from about 160 mm² to about 190 mm², or from about 170 mm² to about 180 mm².

In some aspects, the piezoelectric solid has a volume of from about 100 mm³ to about 200 mm³, or from about 110 mm³ to about 190 mm³, or from about 120 mm³ to about 180 mm³, or from about 130 mm³ to about 170 mm³, or from about 140 mm³ to about 160 mm³, or from about 100 mm³ to about 150 mm³, or from about 110 mm³ to about 140 mm³, or from about 120 mm³ to about 130 mm³, or from about 150 mm³ to about 200 mm³, or from about 160 mm³ to about 190 mm³, or from about 170 mm³ to about 180 mm³.

In another aspect, provided is a device comprising a piezoelectric solid and a miniaturized electronic device; wherein the piezoelectric solid comprises a polymer and metal oxide nanoparticles and has a platonic solid shape; wherein a face of the piezoelectric solid comprises a concentric void; and wherein the miniaturized electronic device is placed inside the concentric void.

In some aspects, the polymer is polyvinyl fluoride, acrylonitrile butadiene styrene, polycarbonate, polyethylene, polypropylene, polyvinyl chloride, or combinations thereof.

In some aspects, the metal oxide is BaTiO₃, Pb(Zrₓ,Ti₁₋ₓ)O₃, PbTiO₃, ZnO, or combinations thereof.

In some aspects, the miniaturized electronic device is a rectifier circuit, a low dropout regulator, a DC-DC regulator, or combinations thereof.

In another aspect, provided is an ultrasonic transducer comprising any of the disclosed devices.

In another aspect, provided is an ultrasonic receiver comprising any of the disclosed devices.

In some aspects, the piezoelectric solid transfers power from an external ultrasonic transducer to the miniaturized electronic device at any angular orientation of the external ultrasonic transducer. This feature of the piezoelectric solid is referred to as “omnidirectionality,” and is primarily conferred by the radial poling of the solid.

In another aspect, provided is an implantable microdevice comprising any of the disclosed ultrasonic receivers, wherein the ultrasonic receiver transfers power to the implantable microdevice via the miniaturized electronic device. In some aspects, the implantable microdevice is a pacemaker, an oxygen generator, an electric stimulator, a chemotherapy agent delivery system, a light delivery system, or combinations thereof.

### Methods

In an aspect, provided is a method of making a piezoelectric solid, comprising: a) preparing a binder solution comprising a polymer and a binder; b) adding metal oxide nanoparticles to the binder solution; c) additively manufacturing the binder solution into a platonic solid shape; d) debinding and sintering the platonic solid to form a piezoelectric solid; and e) poling the piezoelectric solid. [[expand on additive manufacturing in spec, 3D printing, injection molding, etc.]]

As used herein, the term “additive manufacturing” refers to the molding or layering of a material to form a 3D solid, for example, 3D printing or injection molding.

In some aspects, the binder is N,N′dimethylformamide (DMF), dimethyl sulfoxide (DMSO), N-methyl-2-pyrrolidone (NMP), or combinations thereof.

In some aspects, the polymer is polyvinyl fluoride, acrylic, acrylonitrile butadiene styrene, polycarbonate, polyethylene, polypropylene, polyvinyl chloride, or combinations thereof.

In some aspects, the metal oxide is BaTiO₃, Pb(Zrₓ,Ti₁₋ₓ)O₃, PbTiO₃, ZnO, or combinations thereof.

In some aspects, the piezoelectric solid has a tetrahedron shape, a cube shape, an octahedron shape, a dodecahedron shape, or an icosahedron shape.

In some aspects, step e) further comprises: i) creating a concentric void on a face of the piezoelectric solid; ii) placing a first electrode throughout a surface of the piezoelectric solid; iii) placing a second electrode at a base of the void; and iv) inducing an electric field across the piezoelectric solid.

In some aspects, the method further comprises placing a layer of metal epoxy underneath the first electrode and/or the second electrode. This metal epoxy can help direct the electromagnetic current throughout the solid. In some aspects, the metal epoxy is silver epoxy.

In some aspects, the method further comprises placing a miniaturized electronic device inside the concentric void. In some aspects, the miniaturized electronic device is a rectifier circuit, a low dropout regulator, a DC-DC regulator, or combinations thereof.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc) but some errors and deviations should be accounted for. Unless indicated otherwise, parts are parts by weight, temperature is in °C or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1—Materials and Methods

Fabrication of the Barium Titanate Platonic Receiver: FIGS. 1A-1F shows the fabrication process. First, the binder solution containing polyvinyl fluoride (PVDF; Alfa Aesar) and N,N′dimethylformamide (DMF; Thermo Scientific) with a 1:8.8 weight ratio is prepared in a heated water bath (temperature=80 °C) (FIG. 1A). PVDF is slowly dissolved into the DMF solution by gently stirring the mixture for at least 15 minutes. When the PVDF was completely dissolved, BaTiO₃ nanoparticles (size=500 nm; US research nanomaterials) are slowly added until the weight ratio of BaTiO₃ to PVDF/DMF becomes 3.345:1 (FIG. 1B). During the mixing, any clumps are removed by continuous stirring. The mixture is then loaded into a custom-made 3D extrusion printing syringe (10 mL with 600 μm nozzle) for 3D printing Platonic solid shapes (FIG. 1C). After the 3D printing, Platonic solids are baked in an oven (AccuTemp-09, Across International) for 2 hours at 120 °C to evaporate any residual DMF on surfaces (FIG. 1D). To explore the effect of poling direction, especially the radial poling, one group of Platonic solids are modified by creating a concentric void on one face. Note that this concentric cylindrical void can be a compartmental space for circuitry, providing seamless packaging. The Platonic solids are then post-processed through debinding and sintering (FIG. 1E). The debinding process refers to the thermal removal of DMF additives. The sintering process forms a bulk piezoelectric material. They are done sequentially in a tubing furnace (GSL-1600X, MTI Corporation, USA). The debinding is done at 650 °C for 1 hour (ramp rate=5 °C/min), followed by the sintering at 1125 °C for 3 hours (ramp rate=5 °C/min). After the sintering, randomly oriented electric dipoles within the Platonic solids are aligned (FIG. 1F) using the poling process. For the radial poling, Platonic solids with concentric void receive one electrode throughout the surface, and another electrode at the base of the void using a thin layer of silver epoxy (8331, MG Chemical, USA). For the axial poling, the electrodes were placed on the parallel faces of the Platonic solids. It should be mentioned that for the sphere, a 2 mm wide electrical isolated region was created in the equatorial plane during applying the thin layer of silver epoxy on the opposite faces. The poling is done using a high voltage DC supply (230-30R, Spellman, USA) paired with a custom-made poling stage. The poling stage has a bottom copper plate and a spring-loaded needle connector from the top to make firm contact with the electrodes on the Platonic solids. The whole stage is submerged in a silicone oil bath to prevent any electrical conduction through ionized air due to a high-density electric field. The silicone oil bath is then heated up to 120 °C (near Curie temperature of BaTiO₃) and an electric field (>1.0 kV/mm) was induced across the Platonic solids for 2 hours. Lastly, a miniaturized electronic device (e.g., a rectifier circuit and an LED) is placed inside the radial poling Platonic solids and completes the circuit (FIG. 1G). For secure packaging, a droplet of epoxy glue can be added to the void.

Ultrasonic Powering Setup: FIG. 2 show the experimental setup for the resonant frequency study. A sinusoidal signal with various frequencies from 0 to 1 MHz was applied to the Platonic solid through a wideband RF amplifier (1040L, ENI Inc). The acoustic waves generated from the Platonic solids were then captured using a fiber-optic hydrophone (FOH-64, Precision Acoustics, UK).

FIGS. 3A-3C show the experimental setup for the rest of experiments, including orientation misalignment, operation depth, offset, and intensity responses. In distilled water that mimics the soft tissue in terms of acoustic impedance of (~1.5 MRayl²²), a focused ultrasound transducer (TXH-0.67-75, Precision Acoustics, UK) or a planar ultrasound transducer (TXH-500-45, Precision Acoustics, UK) was submerged on the one end. A Platonic solid was then attached to a rod and submerged on the other ends. Using a manipulator (LaserBot, Makerblock) that allows XY movements, the Platonic solids were introduced at various distances, and operation depth, offset, and intensity responses were measured. Note that Z-axis movement was omitted as the offset study is expected to be the same. For the orientation misalignment study, a custom-made rotational platform was added to the manipulator (FIGS. 3B-3C), which could rotate the platonic solids every 3° resolution.

Finite Element Analysis: 3D finite element simulation (frequency domain analysis, 100 k Hz to 1 MHz) was performed in COMSOL Multiphysics (ver. 5.2) software to verify the resonance frequencies. The geometrical shapes were drawn in Fusion 360 (Autodesk) and the 3D models were imported to the simulation environment. The geometries were modeled as BaTiO₃ (both axial and radial poling). The solid mechanics module in the simulation computed the stress distribution in the solids due to the incident load, and the electrostatics interface computed the electric displacement field as well as the potential distributions. The input load (206.6 kPa) during the simulation was kept the same as the incident pressure induced by the acoustic wave during the physical experiments.

Theoretical Calculation of the Angular Response of Radially Poled Platonic Solids Receivers: The voltage output from the radially poled Platonic solids receivers under varying angular misalignments was estimated using the following procedure. Adopting the cylindrical coordinate, the poling direction was set as the radial direction \hat{r}. For simplification, it was assumed that the poling direction is normal to the surface and resulting in a decomposition of the poling vector (FIG. 4A). In this sense, the radial poling is a superposition of axial poling vectors (FIG. 4B). The angular response of each Platonic solid was calculated by the following rational illustrated in FIG. 4C. The ultrasound (denoted by red dashed arrows) is coming in at an angle θ in respect with the surface normal vector of the surface 2; the angle θ was determined by rotating the Platonic solids in respect with the z-axis in 3D CAD program and measuring the projected area of each face (A=A₀ cos θ) when viewing from x-axis, where A is the projected area into the YZ plane and A₀ is the overall area of the face. Assuming a uniaxial poling, the angle between the surface normal vector of the surface 2 and the poling direction a is determined by the geometry of the Platonic solids. After determining the angle for each face at each rotational angle, the charge accumulated in the surface 1 (Q₁) was calculated by summing the induced charges by incoming ultrasound to the surface 1 (Q₁₁) and 2 (Q₁₂), given as below.

Q₁₂=A₀ cos θᵢ[d₃₃|cos(180−(α−θ))|+d₃₁| sin(180−(α−θ))|]

Q₁=ΣQ₁ⱼ=Q₁₁+Q₁₂+ . . .

The total response of the radially poled Platonic solids is given by summing the accumulated charge Q by summing for all faces (note that the counter charge accumulation occurs at the inner cylindrical surface).

Q=ΣQᵢ=ΣΣQᵢⱼ

The open-circuit voltage output can then be obtained by dividing the total charge Q by the capacitance of the solids (V=Q/C). The measured capacitance of the radially poled Platonic solid receivers was similar across all geometry, hence enabling a direct comparison through accumulated charges. The normalized theoretical calculation of the angular responses of the cube, octahedron, and dodecahedron are plotted in FIGS. 9D-9F, respectively, which all show excellent agreement with the experimental results (FIGS. 10A-10F). In addition to the advantage of the radial poling (observed from comparing the two polar plots in FIG. 9D), the Platonic solids with higher order of symmetry clearly show much improved angular response as the normalized angle average response are 0.38, 0.69, and 0.95 for cube, octahedron, and dodecahedron, respectively. Moreover, the calculated response also strongly depends on the geometry with the dodecahedron's maximal output being the largest. The angle averaged response of each Platonic solids is plotted in FIG. 9G, which is largely consistent with the experimental values where the maximum output from dodecahedron was observed. While the sphere will have the best normalized angular response of 1, the maximum value is less than that of dodecahedron. Thus, overall, of the 4 polyhedral investigated in this work, dodecahedron showed the best response both experimentally and theoretically.

Spatial Characterization of Platonic Solids under Ultrasound: Focused ultrasound (FUS) was employed to operate Platonic solids receiver. FUS naturally delivers higher intensity to the specific focal zone,

$${F}_{z} = {NS}_{F}^{2} [ \frac{2}{1 + 0.5 {S}_{F}} ] ,$$

where S_{F} is normalized focal length (=F/N). FIG. 11A depicts the measured output voltages from Platonic solids with respect to the depth. As expected, the outputs were significantly higher near the focal zone (7.5 cm) then rapidly dropped. However, the dodecahedron could sustain the acceptable voltage output (~1 V) up to 14 cm, which is 11.5 dB higher than other Platonic solids.

The voltage outputs in response to different input intensities were also measured. FIG. 11B shows the voltage output in a function of the input intensity. As expected, the dodecahedron delivers a much higher voltage (~8V) at 20 W/cm² of the ultrasonic intensity compared to other Platonic solids. However, the input intensity shall be carefully controlled with the time duration due to the potential bioeffect. While such operation depth is sufficient for most applications, it could be further extended using a regular ultrasound, which can supply moderate ultrasonic intensity over the long-distance, determined by the near field distance N (=D²/4λ where D is the transducer diameter, and λ is the wavelength of the operating frequency). FIG. 11C shows the difference between FUS and regular ultrasound. The FUS induces almost 2.2 times higher voltage output at its focal zone, while the regular ultrasound provides moderate performance over the long-distance (>30 cm).

The offset misalignment is another important factor for wireless ultrasonic powering. It is predetermined by the beam diameter, which is associate with 1.02Fc/fD, where Fis focal length, c is the material sound velocity (~3,800 m/s for FUS transducer that uses PZT as a piezoelectric element), and D is the diameter of the transducer. With the dodecahedron, the measured offset misalignment indicated that the transverse beam could cover up to 2 cm using a 5-cm diameter FUS transducer and 4 cm using a 3-cm diameter planar US transducer (FIG. 11D). The results indicate that the FUS transducer may require an imaging instrument to precisely deliver the power. Alternatively, the planar US transducer could cover a much larger region of operation despite the relatively lower power transfer.

### Example 2—Design of Barium Titanate Platonic Solid for Ultrasonic Powering

Platonic solids are convex polyhedra with faces made of an equivalent regular polygon (Steinhaus, H., 1999. Mathematical snapshots. Courier Corporation). There exist five Platonic solids, namely, the tetrahedron, cube, octahedron, dodecahedron, and icosahedron. Based on regular triangles, the tetrahedron, octahedron, and icosahedron are composed of 4, 8, and 20 faces, respectively. With 6 faces, the cube is the only Platonic solid composed of squares. Similarly, the dodecahedron consists of 12 faces of pentagons. With the highly symmetric shape, tailoring such three-dimensional structural diversity in the ultrasonic receiver can enhance the ultrasonic powering efficiency.

The higher degree Platonic solids such as the octahedron, dodecahedron, and icosahedron increase the probability of aligning the incident ultrasonic waves with the poling direction by a factor of their number of faces (8, 12, and 20 faces, respectively). FIG. 5C illustrates how a dodecahedron (as an example) increases such probability. Furthermore, if the projected faces seen from the incoming ultrasonic waves are combined as an effective ultrasonic receiving area, those higher degree Platonic solids can sustain the relatively consistent effective area even in severe angular misalignments. FIG. 5D summarizes the effective area by taking the projected area at each angular orientation, which is periodically repeated due to the symmetric form factor. For example, the dodecahedron repeats the same effective area every 18° while that of the octahedron is 30°. The periodicity of cube, however, occurs much less frequency at every 45°. The sphere, as a representation of the highest degree of polyhedral, would have an indefinite periodicity.

To test the hypothesis of the potential omnidirectional powering due to geometric effect, three Platonic solids were chosen in increasing order of rotation symmetry in respect to the axial direction: the cube, octahedron (as the regular triangle representative), dodecahedron, and a sphere. The dimension of each Platonic solid was adjusted so that all would have similar overall surface areas; the receiver performance would predominantly be determined by the geometrical shapes. TABLE 1 summarizes the design of Platonic solid, where a is edge length and r is radius. The overall surface areas were equated to 154.04±0.164 mm² with the radius of the sphere to be 3.5 mm, an edge length of 5.07, 6.67, and 2.73 mm for the cube, octahedron, and dodecahedron, respectively.

TABLE 1
Summary of platonic solid receivers dimension.

| | | Surface | | Overall |
|---|---|---|---|---|
| | Number | area | Dimension | surface |
| Platonic solid | of faces | formula | (mm) | area |
| Cube | 6 | 6a² | a = 5.07 | 152.22 |
| Octahedron | 8 | 2\sqrt{3}a² | a = 6.67 | 154.14 |
| Dodecahedron | 12 | 3\sqrt{25 + 10\sqrt{5}}a² | a = 2.73 | 153.87 |
| Sphere | inf | 4πr² | r = 3.5 | 153.94 |

Barium titanate (BaTiO₃) is selected as the materials for the Platonic solid ultrasonic receiver due to processing flexibility, good piezoelectric and mechanical properties (summarized in TABLE 2), and its biocompatibility. It should also be noted that the purpose of selecting BaTiO₃ (a lead-free material) is to introduce a non-toxic implantable piezoelectric source for implantable devices. Although the coupling efficiency is compromised slightly when compared to traditional lead-based piezoelectric materials (i.e., Pb(Zr,Ti)O₃ (PZT), PbTiO₃ (PTO)), the goal is to demonstrate that it is possible to transmit power even with a lower coupling coefficient, especially when the transducer shape is optimized.

TABLE 2
Summary of BaTiO₃ material properties (Berlincourt, D., et al.,
1958. Elastic and piezoelectric coefficients of single-crystal
barium titanate. Physical Review 111, 143) (Frey, M., et al.,
1996. Grain-size effect on structure and phase transformations
for barium titanate. Physical Review B 54, 3158)

| Properties | Value |
|---|---|
| Density (g/cm³) | 5.80 |
| Young's modulus (GPa) | 67 |
| ε′ | 1680 |
| kₜ | 0.26 |
| d₃₃ (pC/N) | 160-350 |
| g₃₃ (Vm/N) | 11.4 |
| Acoustic velocity (m/s) | 1790 |

Adopting the fabrication processes reported in (Park, M., et al., 2020. Human Oral Motion-Powered Smart Dental Implant (SDI) for In Situ Ambulatory Photobiomodulation Therapy. Adv. Healthcare Mater. 9, 2000658), Platonic solid receiver prototypes were fabricated (FIG. 6A). The morphological features and grain sizes were observed using a scanning electron microscope (SEM; Quanta FEG 250, FEI). FIGS. 6B-6C shows SEM images before and after the post-processing. As seen, the BaTiO₃ nanoparticles merged into large grain ceramic. To obtain information on the crystal structures and orientation, X-ray diffraction (XRD) was performed on the fabricated BaTiO₃ ceramic before and after poling (FIG. 6D) (D8 advance powder XRD, Bruker with Cu-Kα radiation (λ=1.5418 Å, 40 kV, 40 mA) operating in the reflection mode). The diffraction peak positions were in good agreement with the literature (Acosta, M., et al., 2017. BaTiO₃-based piezoelectrics: Fundamentals, current status, and perspectives. Applied Physics Reviews 4, 041305) (Roberts, S., 1947. Dielectric and piezoelectric properties of barium titanate. Physical Review 71, 890). The tetragonal phase of BaTiO₃ ceramic was confirmed by the peak splitting at 20 near 22° and 45°. Furthermore, the enhancement of peak ratios of (001)/(100) and (002)/(200) was observed after poling indicating the piezoelectric domains are aligned along the poling direction of (001).

### Example 3—Omnidirectional Wireless Ultrasonic Powering

Although the high rotational symmetry of the Platonic solids would permit a higher chance of angular alignments between the effective receiving surface and the incident ultrasound, this advantage can be mitigated by poling along a single axis. Thus, to take full advantage of the high symmetry of Platonic solids enabled by the versatile 3D printing technique, a study was conducted which explored the effect of poling directions: a radial or an axial (FIGS. 7A-7E). To enable the radial poling, the Platonic solid was hollowed out up to the center and a poling process was performed; applying a strong electrical field (>1 kV/mm) in heated silicone oil (>120 °C) for over 4 hours. Thus, the poling direction is radial spreading from the inner cylindrical surface to the outer surface of the Platonic solids corresponding to the radial direction of the cylindrical coordinate (FIG. 7A). Note that the axial poling was done through two parallel planes of electrodes (FIG. 7B).

Firstly, the radial poling affected the resonant frequency, which could be measured by driving the Platonic solids as a source of acoustic source in the distilled water by connecting to a signal generator. FIG. 7C summarizes the experimental and simulation results of each Platonic solids. The Platonic solids that were poled in an axial direction showed average resonant frequency of 370±35 kHz (f_{r,axial, Cube}=320 KHz, f_{r,axial, Octahedron}=400 kHz, f_{r, axial, Dodecahedron}=370 kHz, f_{r.axial. Sphere}=390 kHz). Under radial poling direction, these resonant frequencies shifted to a higher region of 502.5±47 kHz due to the higher-order resonant vibration modes (f_{r,radial. Cube}=490 kHz, f_{r, radial, Octahedron}=460 kHz., f_{r,radial, Dodecahedron}=570 kHz, f_{r,radial, Sphere}=490 kHz). The measured resonant frequencies also showed excellent agreement with the finite element analyses (Multiphysics COMSOL simulations), confirming the successful poling in the radial direction. FIGS. 4A-4D show the complete results of the COMSOL simulation.

Next, the advantage of the radial poling for omnidirectional powering is validated. The voltage outputs from two identical cubical transducers but one with axial poling and the other with radial poling were measured at different misalignment angles (FIGS. 7D-7E; FIGS. 8A-8B shows corresponding XY plots). The normalized polar plot clearly demonstrates the advantage of radial poling over axial poling. While the stiff decrease in the open circuit voltage (V_{oc}) is observed in axially poled transducer especially pronounced around 90° (FIG. 7D), the voltage output from the radially poled transducer is more resilient under angular misalignments (FIG. 7E).

This dramatic enhancement in omnidirectionality is clearly due to the radial poling; to further confirm this, theoretical calculations were carried out. Briefly, the induced charge density at the surface of the electrode is related to the stress by piezoelectric coefficients. Neglecting the shear components, the induced surface charges by a single plane whose surface normal vector is at angle θ with respect to the incoming ultrasound can be formulated (FIGS. 9A-9G). Summing the accumulated charges on all surfaces, the output generated by the piezoelectric Platonic solids can be estimated as:

$$Q = \sum {A}_{0} cos {θ}_{i} [ {d}_{33} \vert cos ( 180 - ( {α}_{i} - {θ}_{i} ) ) \vert + {d}_{31} \vert sin ( 180 - ( {α}_{i} - {θ}_{i} ) ) \vert ] = \sum {A}_{0} cos {θ}_{i} {d}_{{eff}_{i}}$$

where A₀ is an effective area, α is a poling direction determined by the geometry of the Platonic solids, θ is the acoustic intensity of the incident wave, and d_{eff} is the effective piezoelectric coefficient enabled by Platonic solid geometric effect. The validity of the theoretical calculation is confirmed by the excellent qualitative and quantitative agreements with the measurements (FIGS. 7D-7E, FIGS. 10A-10F). Overall, the angle-averaged radially poled cube output is about 40% while that of the axially poled cube is only about 20% (FIGS. 7D-7E).

Having established the advantage of the radially polarized Platonic solids, now the study proceeded to explore the effects of the degree of rotational symmetry on omnidirectionality. To evaluate the enhancement in the omnidirectionality of the ultrasonic powering by the introduction of the higher order Platonic solids, the BaTiO₃ Platonic solids were excited by focused ultrasound with and without intentional misalignment (FIGS. 8A-8C). With the Platonic receiver is placed 7.5 cm away from the transducer (focal zone), the ultrasonic transducer was operated with 10-cycles of sinusoidal signals that are amplified through an RF amplifier (fᵣ~502.5 kHz; duty cycle=1.4%, I_{SPTA}=1.3 W/cm²). This fixed the direction of the incoming ultrasound to the x-direction. The waveform shown in FIG. 8D confirms the input signal (on the left) and received signal (on the right); the time delay (~48 μs) also indicates the 7.5 cm in the distance between the transducer and receiver. A Platonic solid receiver is then modulated for comprehensive studies of alignment degrees of freedom.

The voltage outputs measured at different axial angles clearly validated the omnidirectional ultrasonic powering of higher order Platonic solids (FIGS. 10A-10F; FIGS. 8A-8E shows XY plots). The cube showed significant voltage losses at 45°, 135°, 225°, and 315° when one of the edges is exposed to the incoming waves; 75% of reduction compared to its maximum output (V_{Cube, max}=1.07 V, V_{Cube, min}=0.27 V) (FIG. 8A, FIG. 10A). The octahedron showed a similar amount of voltage drops at the edges; however, it generally exhibited the highest voltage output more often due to its higher periodicity of 30° (FIG. 8C, FIG. 10B; note that the measurement of 140° was repeated to create the polar plot due to the extruding electrical contact at one side of the octahedron). Furthermore, the maximum reduction in the voltage output was slightly less as 68% to its maximum output (V_{Octahedron, max}=2.22 V, V_{Octahedron, min}=0.71 V). The results clearly show that the Platonic solids with the higher order of symmetry significantly improved angular response. The improvement was even more prominent in the dodecahedron that has 12 faces with symmetricity every 18°; almost consistent voltage outputs were observed across all angles (V_{Dodecahedron, max}=4.17 V, V Dodecahedron, min=2.95 V) (FIG. 8D, FIG. 10C; to obtain the polar plot, the measurement data was drift-compensated) with only a moderate output voltage reduction (29%). Lastly, the sphere shows almost omnidirectional powering (FIG. 8E, FIG. 10D). However, the overall voltage outputs were only comparable to that of the octahedron (V_{Sphere, max}=2.11 V). It is because the curved surface of the sphere may have skipped the incoming ultrasonic waves instead of striking. The minimum voltage, V_{Sphere. min}, of 1.53 V was also observed which is a 27% drop compared to the maximum likely due to the sample imperfection. Based on the orientation results, the study could conclude that the dodecahedron can deliver a relatively higher power output with excellent omnidirectional responses. All other spatial characterizations, including operation depth, offset, and comparison to the planar ultrasound transducer are discussed in the Supplementary Information (FIGS. 11A-11D).

Lastly, the current-voltage (IV) characteristics (FIG. 10E) and power density (FIG. 10F) of all Platonic solid receivers were measured with various resistive loads. As expected, it was observed that the dodecahedron could produce the highest power output (16.8 mW; η=4.90%); P_{out. cube}=2.5 mW (n=0.73%), P_{out. octahedron}=6.8 mW (n=1.98%), and P_{out, sphere}=6.7 mW (n=1.95%). Although such low power transfer efficiencies may not be significant, it is a threefold improvement compared to other studies with the PZT, which has a much higher electromechanical coefficient. Here, the capacitances of the radially poled Platonic solids were about one order of magnitude greater than those of axially poled ones (average C_{Axial} was 22.26 pF and average C_{Radial} was 272.21 pF, measured with an impedance analyzer (E5061B, Keysight)); thus, for the same voltages, the overall power generated from the radially poled Platonic solids are much greater than the axially poled ones. Considering the millimeter scale, true omnidirectional operation, and lead-free, the reported radially poled Platonic solids are indeed better performance than others.

### Example 4—Radially Poled Platonic Solid for Implantable Micro Light Source

Thus far, the advantages of radially poled Platonic solids with high rotational symmetry have been explored, which could drastically improve the ultrasonic powering through higher symmetry in its shape in terms of omnidirectionality and superior power conversion efficiency. Another study was conducted which explored a seamless packaging application using the radially poled Platonic solid ultrasonic receiver, which can potentially address outstanding challenges in developing implantable devices—powering and packaging.

The study utilized the concentric cylindrical void for the radial poling. In addition, the void can also double as the compartmental space for circuitry to enable the radially poled Platonic solid as a packaging material for the small implants in addition to serving as the power source. In this sense, barium titanate shell is especially suitable since it offers excellent biocompatibility and mechanical properties (Acosta, M., et al., 2017. BaTiO3-based piezoelectrics: Fundamentals, current status, and perspectives. Applied Physics Reviews 4, 041305) (Luo, C., et al., 2018. A Flexible Lead-Free BaTiO₃/PDMS/C Composite Nanogenerator as a Piezoelectric Energy Harvester. Energy Technol. 6, 922-927). Lastly, the 3D printability of the ultrasonic receiver presented in this work allows the customization of the dimensions of the Platonic solid packaging. As a proof-of-concept, an implantable micro-light source (μLight) was fabricated, which can deliver light to deeply seated tumors for photodynamic therapy (Kim, A., et al., 2019. An Implantable Ultrasonically-Powered Micro-Light-Source (μLight) for Photodynamic Therapy. Sci Rep 9, 1395).

To demonstrate the Platonic solid-packaged μLight device, firstly a radially poled Platonic solid receiver was fabricated, which offers the combination of omnidirectionality and high-power output. An LED and the powering circuitry were then placed inside the cylindrical void of 2 mm in diameter and 2 mm in height (FIG. 12A). The overall dimension of the device was 155 mm³ in volume, small enough to fit into 10-gauge biopsy needle. In FIG. 12B, the ultrasonic excitation of the implantable light source is shown; the external transducer was set to generate the ISPTA of 15 mW/cm². The bright LED illumination was observable. Blue (465 nm) and red (625 nm) LEDs were tested to demonstrate the versatility. Notably, the μLight could deliver robust light irradiance regardless of the transducer-receiver orientation. The μLight was also tested using ex vivo porcine tissue. For the ex vivo validation, the μLight was implanted in the porcine tissue by first creating a small puncture using a biopsy needle (10G, diameter=3.4 mm) (FIG. 13). The device was then pushed into the focal point of the focused ultrasound. The position of the μLight was confirmed by ultrasonic imaging system as shown in FIGS. 12C-12D (iQ+, Butterfly Network Inc). At the implantation site, the focused ultrasound was delivered with the ISPTA of 15 mW/cm² with ~2% duty cycle (bursting 10 cycles of 500 kHz sinusoidal signal for 1 msec). Clear illumination of the LED was observed (FIG. 12E). While the μLight was placed at the focal point of the ultrasound, the angular alignment was left at random. Thus, the successful operation of the μLight in porcine tissue is another validation of the omnidirectionality enabled by the radially poled Platonic solids.

### DISCUSSION

In this work, a 3D printed barium titanate Platonic solid ultrasonic receiver is presented as an enabling factor for omnidirectional ultrasonic powering, which can be a translational solution for many implantable devices. The combination of radial poling and highly symmetric transducer geometry was explored as an omnidirectional ultrasonic receiver. This work showed the Platonic solids that have a highly symmetric geometry could increase the probability of receiving ultrasonic waves coming in from any random direction. When the radial direction poling is paired, the Platonic solids exhibited superior power transfer efficiency with true omnidirectionality. Interestingly, the Platonic solids with the concentric cylindrical void that was created for the radial poling could be utilized as a seamless packaging for circuitry, addressing outstanding challenges in developing implantable devices in powering and packaging.

1. A piezoelectric solid comprising a polymer and metal oxide nanoparticles, wherein the piezoelectric solid has a platonic solid shape.

2. The piezoelectric solid of claim 1, wherein the metal oxide is BaTiO₃, Pb(Zrₓ,Ti₁₋ₓ)O₃, PbTiO₃, ZnO, or combinations thereof.

3. The piezoelectric solid of claim 1, wherein the polymer is a thermoplastic.

4. The piezoelectric solid of claim 3, wherein the polymer is polyvinyl fluoride, acrylonitrile butadiene styrene, polycarbonate, polyethylene, polypropylene, polyvinyl chloride, or combinations thereof.

5. The piezoelectric solid of claim 1, wherein the piezoelectric solid has a tetrahedron shape, a cube shape, an octahedron shape, a dodecahedron shape, or an icosahedron shape.

6. The piezoelectric solid of claim 5, wherein the piezoelectric solid is radially poled.

7. The piezoelectric solid of claim 1, further comprising a concentric void on a face of the piezoelectric solid.

8. A device comprising a piezoelectric solid and a miniaturized electronic device,
wherein the piezoelectric solid comprises a polymer and metal oxide nanoparticles and has a platonic solid shape;
wherein a face of the piezoelectric solid comprises a concentric void; and
wherein the miniaturized electronic device is placed inside the concentric void.

9. The device of claim 8, wherein the polymer is polyvinyl fluoride, acrylonitrile butadiene styrene, polycarbonate, polyethylene, polypropylene, polyvinyl chloride, or combinations thereof.

10. The device of claim 8, wherein the metal oxide is BaTiO₃, Pb(Zrₓ,Ti₁₋ₓ)O₃, PbTiO₃, ZnO, or combinations thereof.

11. The device of claim 8, wherein the miniaturized electronic device is a rectifier circuit, a low dropout regulator, a DC-DC regulator, or combinations thereof.

12. An ultrasonic transducer comprising the device of claim 8.

13. An ultrasonic receiver comprising the device of claim 8.

14. The ultrasonic receiver of claim 13, wherein the piezoelectric solid transfers power from an external ultrasonic transducer to the miniaturized electronic device at any angular orientation of the external ultrasonic transducer.

15. An implantable microdevice comprising the ultrasonic receiver of claim 13, wherein the ultrasonic receiver transfers power to the implantable microdevice via the miniaturized electronic device.

16. A method of making a piezoelectric solid, comprising:
a) preparing a binder solution comprising a polymer and a binder;
b) adding metal oxide nanoparticles to the binder solution;
c) additively manufacturing the binder solution into a platonic solid shape;
d) debinding and sintering the platonic solid to form a piezoelectric solid; and
e) poling the piezoelectric solid.

17. (canceled)

18. (canceled)

19. The method of claim 16, wherein the binder is N,N′dimethylformamide (DMF), dimethyl sulfoxide (DMSO), N-methyl-2-pyrrolidone (NMP), or combinations thereof.

20. (canceled)

21. The method of claim 16, wherein step e) further comprises:
i) creating a concentric void on a face of the piezoelectric solid;
ii) placing a first electrode throughout a surface of the piezoelectric solid;
iii) placing a second electrode at a base of the void; and
iv) inducing an electric field across the piezoelectric solid.

22. The method of claim 21, further comprising placing a layer of metal epoxy underneath the first electrode and/or the second electrode.

23. The method of claim 21, further comprising placing a miniaturized electronic device inside the concentric void.

24. (canceled)
